(19) United States
(12) Patent Application Publication
(10) Pub. No.: US 2024/0382654 A1
(43) Pub. Date: Nov. 21, 2024

(54) CHEMICAL CROSS-LINKING BASED POROUS MATERIAL FOR TISSUE REGENERATION AND HEMOSTASIS COMPRISING EXTRACELLULAR MATRIX OF DECELLULARIZED KIDNEY TISSUE AND PREPARATION METHOD THEREOF

(71) Applicants: POSTECH Research and Business Development Foundation, Pohang-si (KR); INDUSTRY-ACADEMIC COORPERATION FOUNDATION DAEGU HAANY UNIVERSITY, Gyeongsan-si (KR)

(72) Inventors: Jae Yun KIM, Seoul (KR); Tugce SEN, Seoul (KR); Jae Yeon LEE, Gyeongsan-si (KR); Dong-Woo CHO, Seoul (KR)

(73) Assignees: POSTECH Research and Business Development Foundation, Pohang-si (KR); INDUSTRY-ACADEMIC COORPERATION FOUNDATION DAEGU HAANY UNIVERSITY, Gyeongsan-si (KR)

(21) Appl. No.: 18/663,594

(22) Filed: May 14, 2024

(30) Foreign Application Priority Data
May 15, 2023 (KR) 10-2023-0062357
Feb. 15, 2024 (KR) 10-2024-0021658

(51) Int. Cl. A61L 27/36; A61L 27/54; A61L 27/56

(52) U.S. Cl. CPC A61L 27/3691; A61L 27/3633; A61L 27/3687; A61L 27/54; A61L 27/56; A61L 2400/04; A61L 2430/34; A61L 2430/40

(57) ABSTRACT

The present invention relates to a chemical cross-linking based porous material for tissue regeneration and hemostasis, including an extracellular matrix of decellularized kidney tissue, and a method for production thereof. The porous material including an extracellular matrix of decellularized kidney tissues according to the present invention forms a chemical cross-linking bond, while the final product does not contain any existing cross-linking agent component, whereby the porous material can be utilized in biocompatible substances. Further, the porous material of the present invention does not only have rapid hemostasis but also delays degradation in a long term to thus exhibit tissue regeneration effects, so that the porous material is expected to be usefully applicable for treatment of different diseases due to tissue damage in a variety of tissues, or for hemostasis and tissue regeneration at removal of tissues or incision operation.

### CROSS REFERENCE TO RELATED APPLICATIONS

This application claims priority to Korean Patent Application No. 10-2023-0062357 filed on May 15, 2023 and Korean Patent Application No. 10-2024-0021658filed on Feb. 15, 2024, the entire disclosures of which are incorporated herein by reference.

### FIELD OF INVENTION

The present invention relates to a chemical cross-linking based porous material for tissue regeneration and hemostasis, including extracellular matrix of decellularized kidney tissue, and a preparation method thereof.

### BACKGROUND OF INVENTION

In the modern age, surgical operations for treatment of different tissues and organ damage are being increased rapidly through advanced modern medical technology. Bleeding caused by light or minor abrasions, cutting wounds and puncture wounds or during simple surgical procedures can be sufficiently controlled by hemostasis within the body. However, in the case of excessive bleeding in medical emergencies such as big accidents, surgical operation, etc., blood loss going over 40% may lead to death and thus hemostatic treatment is essentially required.

In recent years, hemostatics based on a variety of material compositions and processing forms are generally used in an operating room. Among them, a hemostatic in the form of sponge aims to rapidly absorb and control blood bleeding in a large-scale wound.

Hemostatics using gelatin or collagen have been mostly used, however, the sponge based on gelatin or collagen was not confirmed to be beneficial in an aspect of regeneration. On the contrary, glomerulu necrosis occurrence has been reported in some clinical results. Further, to provide physical/chemical micro-environment to cells with a material only for the purpose of hemostasis in consideration of a self-recovery process of tissues (migration of reproduction-related cells into affected area and secretion of extracellular matrix, tissue formation) has never been published, in addition, there is no chance to tissue regeneration due to remarkably rapid degradation of the material

Meanwhile, the extracellular matrix of decellularized tissue may include different proteins, growth factors, cytokine, etc., therefore, have superior properties compared to single substances (collagen, hyaluronic acid, chitosan, etc.), in terms of biocompatibility.

Accordingly, the present inventors have produced an extracellular matrix of decellularized kidney tissues with biocompatibility using a porous substance, and intended to utilize the above extracellular matrix not only for hemostasis at a bleeding site during surgical operation but also for tissue regeneration at a transplanted area if the porous substance is transplanted.

### PRIOR ART LITERATURE

### Patent Document

Korean Patent Laid-Open Publication No. 10-2023-0164270

### SUMMARY OF INVENTION

### Technical Problem to be Solved

The present inventors have studied to solve a problem of existing hemostatics which are used only for hemostasis and do not exhibit tissue regeneration effects due to high degradation rate and, as a result of the studies, have found that, when a porous substance is prepared by reacting an extracellular matrix of decellularized kidney tissues with EDC/NHS cross-linking agent, chemical cross-linkage may be formed while any existing cross-linking agent component is not contained in the final product, whereby it is very beneficially used in biocompatible materials, stops bleeding quickly, and delays degradation in the long term to thus exhibit tissue regeneration effects. Therefore, on the ground described above, the present invention has been completed.

Accordingly, an object of the present invention is to provide a method for preparation of a porous material for tissue regeneration and hemostasis, comprising an extracellular matrix of decellularized kidney tissues.

Further, another object of the present invention is to provide a porous material for tissue regeneration and hemostasis, comprising an extracellular matrix of decellularized kidney tissues; a composition or kit for tissue regeneration and hemostasis, comprising the same; or a pharmaceutical composition for prevention or treatment of tissue damage-based diseases, comprising the same.

However, technical tasks to be achieved by the present invention are not particularly limited to the above mentioned objects, and other tasks not mentioned herein will be clearly understood by those skilled in the art, to which the present invention pertains, from the following detailed description.

### Technical Solution

In order to accomplish the above objects, the present invention provides a method for preparation of a porous material for tissue regeneration and hemostasis, comprising the following steps:
(a) cutting a separated kidney tissue into sections and washing the same;
(b) treating the sections with a surfactant and agitating the same;
(c) removing DNA component residue within the kidney tissue;
(d) lyophilizing the kidney tissue free of the DNA component to thus obtain an extracellular matrix of decellularized kidney tissue;
(e) mixing the extracellular matrix of the decellularized kidney tissue with a cross-linking agent, followed by stirring and freezing the mixture; and
(f) washing the frozen mixture and lyophilizing the same to thus produce a porous material including the extracellular matrix of the decellularized kidney tissue.

In an embodiment of the present invention, the porous material may be a sponge, but is not particularly limited thereto.

In another embodiment of the present invention, the step (b) may be a step of treatment together with sodium chloride, but is not limited thereto.

In another embodiment of the present invention, the cross-linking agent may be one or more selected from the group consisting of 1-ethyl-3-(3-dimethylaminopropyl) carbodiimide hydrochloride (EDC) and N-hydroxysucinimide (NHS), but is not limited thereto.

In another embodiment of the present invention, the freezing in the step (e) may be conducted at −40 to −10° C. for 12 to 36 hours, but is not limited thereto.

In another embodiment of the present invention, the cross-linking agent may be used as a mixture of EDC solution: NHS solution=2 to 6:1 mole ratio, but is not limited thereto.

In another embodiment of the present invention, the cross-linking agent may be used as a mixture of EDC solution: NHS solution=1:0.5 to 2 mass ratio, but is not limited thereto.

In another embdiment of the present invention, in the step (e), a solution of the extracellular matrix of decellularized kidney tissue: the cross-linking agent solution=4 to 8:1 mass ratio may be admixed, followed by agitating the same, but is not limited thereto.

In another embodiment of the present invention, the porous material may be used for reproduction and hemostasis of one or more tissues selected from the group consisting of kidney tissue, liver tissue, heart tissue, intestinal tissue, skin tissue, stomach tissue, pancreas tissue, brain tissue, thyroid tissue, lung tissue, and spleen tissue, but is not limited thereto.

Further, the present invention provides a porous material for tissue regeneration and hemostasis, comprising an extracellular matrix of decellularized kidney tissue,
wherein the extracellular matrix of the decellularized kidney tissue has a structure consisting of a chemical cross-linking bond between proteins.

According to an embodiment of the present invention, the chemical cross-linking bond may be formed by one or more cross-linking agents selected from the group consisting of 1-ethyl-3-(3-dimethylaminopropyl) carbodiimide hydrochloride (EDC) and N-hydroxysuccinimide (NHS), but is not limited thereto.

According to another embodiment of the present invention, the porous material may have one or more among the following activities, but is not limited thereto:
(i) reduction of a degradation rate;
(ii) hemostasis (bleeding stop) in damaged site of tissue;
(iii) tissue regeneration; and
(iv) increase in proportion of M2 macrophages.

In another embodiment of the present invention, the porous material may be used for transplantation in vivo or as an external skin preparation, but is not limited thereto.

In another embodiment of the present invention, the porous material may be produced in one or more forms selected from the group consisting of gauze, sheet, patch and pad.

Further, the present invention provides a kit for tissue regeneration and hemostasis, comprising the porous material for tissue regeneration and hemostasis.

Further, the present invention provides a pharmaceutical composition for prevention or treatment of diseases caused by tissue damage, comprising the porous material for tissue regeneration and hemostasis as an active ingredient.

According to one embodiment of the present invention, the disease may be any disease due to damage in one or more tissues selected from the group consisting of kidney tissue, liver tissue, heart tissue, intestinal tissue, skin tissue, stomach tissue, pancreas tissue, brain tissue, thyroid tissue, lung tissue, and spleen tissue, but is not limited thereto.

According to another embodiment, the composition may be used for medical treatment of tissue damage sites, or for hemostasis and tissue regeneration in the case of tissue ablation or incision operation, but is not limited thereto.

Further, the present invention provides a method for prevention or treatment of a disease caused by tissue damage, comprising treating a subject in need of treatment with a composition comprising the porous material for tissue regeneration and hemostasis as an active ingredient.

Further, the present invention provides a use of a composition comprising the porous material for tissue regeneration and hemostasis as an active ingredient, the use comprising prevention or treatment of diseases caused by tissue damage.

Still further, the present invention provides a use of the porous material for tissue regeneration and hemostasis, the use comprising production of a preparation for prevention or treatment of diseases caused by tissue damage.

### Effect of Invention

The porous material including an extracellular matrix of decellularized kidney tissues according to the present invention forms a chemical cross-linking bond, while the final product does not contain any existing cross-linking agent component, whereby the porous material may be utilized in biocompatible substances. Further, the porous material of the present invention does not only have rapid hemostasis but also delays degradation in a long term to thus exhibit tissue regeneration effects, so that the porous material is expected to be usefully applicable for treatment of different diseases due to tissue damage in a variety of tissues, or for hemostasis and tissue regeneration at removal of tissues or incision operation.

### BRIEF DESCRIPTION OF DRAWINGS

FIG. 1 is a schematic view illustrating a process for production and application of kdES according to one embodiment of the present invention.

FIG. 2A illustrates the morphology of kdES (E12N3, E24N6, and E48N12) prepared at different EDC/NHS concentrations (upper) according to one embodiment of the present invention; and the microstructure of kdES confirmed through scanning electron microscope (SEM) as compared to commercially available gelatin sponge (lower).

FIG. 2B illustrates the result of determining the cross-linking degree of kdES (E12N3, E24N6, and E48N12) prepared at different EDC/NHS concentrations according to one embodiment of the present invention.

FIG. 2C illustrates the result of analyzing the infrared ray transmittance of each of kdES (E12N3, E24N6, and E48N12) and kdECM prepared at different EDC/NHS concentrations according to one embodiment of the present invention, through Fourier transform infrared spectroscopy.

FIG. 3A illustrates the result of confirming the coefficient of swelling of each of kdES (E12N3, E24N6, and E48N12) prepared at different EDC/NHS concentrations according to one embodiment of the present invention and the gelatin sponge.

FIG. 3B illustrates the result of confirming swelling dynamic of each of kdES (E12N3, E24N6, and E48N12) prepared at different EDC/NHS concentrations according to one embodiment of the present invention and the gelatin sponge.

FIG. 4 illustrates the result of determining the degradation rate of each of kdES (E12N3, E24N6, and E48N12) prepared at different EDC/NHS concentrations according to one embodiment of the present invention and the gelatin sponge in ex vivo environment.

FIG. 5 illustrates the result of confirming cell viability and growth rate by each of kdES (E12N3, E24N6, and E48N12) prepared at different EDC/NHS concentrations according to one embodiment of the present invention and the gelatin sponge.

FIG. 6 illustrates cell cytolysis rate in blood (upper) and images of samples (lower), when the blood was treated with each of kdES (E12N3, E24N6, and E48N12) prepared at different EDC/NHS concentrations according to one embodiment of the present invention and the gelatin sponge.

FIG. 7A illustrates the result of confirming blood coagulation extent per time when treating the blood with kdES according to one embodiment of the present invention and the gelatin sponge, as compared to single processing of the blood.

FIG. 7B illustrates the result of SEM images of kdES and the gelatin after conducting whole cell adhesion according to one embodiment of the present invention

FIG. 8 illustrates the proteomic analysis result of kdES according to one embodiment of the present invention.

FIG. 9A is a schematic view illustrating kidney incision of a rat and application of sponge including kdES according to one embodiment of the present invention.

FIG. 9B illustrates the result of confirming effects of reduction of hemostasis time by transplantation of kdES and gelatin sponge in the kidney incision rat according to one embodiment of the present invention.

FIG. 9C illustrates the result of confirming effects of reduction of blood loss by transplantation of kdES and gelatin sponge in the kidney incision rat according to one embodiment of the present invention.

FIG. 10A illustrates the result of confirming wound restoration effects by histomorphological inspection through H&E, MT, CD44, and PCNA staining on day 3 and day 28 after transplantation of kdES or gelatin sponge in the kidney incision rat according to one embodiment of the present invention.

FIG. 10B illustrates enlarged H&E staining result (upper in FIG. 10B), and H&E staining result and quantification of nephric tubule necrosis (lower in FIG. 10B) on day 3and day 28 after transplantation of kdES or gelatin sponge in the kidney incision rat according to one embodiment of the present invention.

FIG. 11 illustrates the result of confirming macrophage polarization in ex vivo environment by kdES and gelatin sponge according to one embodiment of the present invention.

FIG. 12 illustrates the result of confirming degradation extent of kdES and healing condition as time passed such as 3, 14 and 28 days after transplantation of kdES or porous kdECM in kidney incision rats according to one embodiment of the present invention.

### BEST MODE

In one embodiment of the present invention, a sponge of extracellular matrix of decellularized kidney tissues based on chemical cross-linking type amide bond (see Example 1), and it was confirmed that, when a concentration of EDC/NHS solution used for preparation of kdES increases, the cross-linking degree is increased while having low infrared ray transmittance (see Example 2).

In another embodiment of the present invention, as a result of analyzing the coefficient of swelling and swelling dynamics of kdES, it was confirmed that as a concentration of the cross-linking agent increases, absorbance tends to decrease (see Example 3).

In another embodiment of the present invention, as a result of determining a degradation rate of kdES through biodegradation experiments in ex vivo environment, it could be confirmed that the kdES group has more delayed degradation as compared to gelatin, and as a concentration of EDC/NHS cross-linking agent increases, the degradation is more delayed (see Example 4).

In another embodiment of the present invention, as a result of analyzing cell viability and growth rate according to kdES treatment in ex vivo environment, cell viability was maintained to more than 90% and, in the case of lowest concentration of the cross-linking agent, highest cell growth rate was confirmed (see Example 5).

In another embodiment of the present invention, as a result of assessing blood compatibility (or biocompatibility), when the blood was treated on the gelatin sponge, a cytolysis rate was higher than the kdES group. Further, it was confirmed that one with the lowest cross-linking agent concentration among kdES exhibits the lowest cytolysis extent (see Example 6).

In another embodiment of the present invention, as a result of hemostasis ability

of kdES, the kdES treatment group showed rapidly increased hemostasis effects within 5minutes (see Example 7).

In another embodiment of the present invention, as a result of proteomic analysis of kdES, among functions of various types of proteins, hemostasis ability and tissue regeneration-related functions were determined (see Example 8).

In another embodiment of the present invention, as a result of assessing in vivo hemostasis ability of kdES, it was confirmed that, when kdES is transplanted, a hemostasis time and blood loss were considerably reduced (see Example 9).

In another embodiment of the present invention, as a result of histological assessment of post-transplantation process of kdES, it was confirmed that the damage did not become worsen around kdES transplanted site while tissues get better (see Example 10).

In another embodiment of the present invention, as a result of confirming macrophage polarization in ex vivo environment by kdES through flow cytometry, the proportion of M2 cells (CD206+) was considerably high and given increased weight over time. Accordingly, it was confirmed that, when kdES is transplanted in a body, M2 cells are given increased weight, and therefore, possibly get involved in reproduction and healing (see Example 11).

In a further embodiment of the present invention, as a result of degradation extent of kdES after kdES transplantation in kidney incision rat, it was confirmed that the kdES transplanted group show delayed degradation compared to porous kdECM (see Example 12).

Hereinafter, the present invention will be described in detail.

The present invention provide a method for production of a porous material for tissue regeneration and hemostasis, comprising the following steps:
(a) cutting a separated kidney tissue into sections and washing the same;
(b) treating the sections with a surfactant and agitating the same;
(c) removing DNA component residue within the kidney tissue;
(d) lyophilizing the kidney tissue free of the DNA component to thus obtain an extracellular matrix of decellularized kidney tissue;
(e) mixing the extracellular matrix of the decellularized kidney tissue with a cross-linking agent, followed by stirring and freezing the mixture; and
(f) washing the frozen mixture and lyophilizing the same to thus produce a porous material including the extracellular matrix of the decellularized kidney tissue.

In the present invention, the “porous material” means a substance having lots of pores in a structure of the substance, wherein the shape of pore may be different such as sphere, oval, irregular rod shapes, etc. In general, the pore is an empty space occurring due to expansion of air bubbles (or foam) when the material is mold-formed. In the present invention, the porous material may be a sponge, but is not limited thereto.

In the present invention, the “sponge” means a porous net made of bio-material capable of absorbing body fluid including blood when the sponge is applied to a damaged site. In the present invention, the porous material may be produced in the form of, for example, gauze, sheet, patch, or pad, but is not limited thereto. In the present invention, the porous material may be used for reproduction and hemostasis of one or more tissues selected from the group consisting of kidney tissue, liver tissue, heart tissue, intestinal tissue, skin tissue, stomach tissue, pancreas tissue, brain tissue, thyroid tissue, lung tissue, and spleen tissue, but is not limited thereto.

Further, the present invention provide a method for production of a sponge for tissue regeneration and hemostasis, comprising the following steps:
(a) cutting a separated kidney tissue into sections and washing the same;
(b) treating the sections with a surfactant and agitating the same;
(c) removing DNA component residue within the kidney tissue;
(d) lyophilizing the kidney tissue free of the DNA component to thus obtain an extracellular matrix of decellularized kidney tissue;
(e) mixing the extracellular matrix of the decellularized kidney tissue with a cross-linking agent, followed by stirring and freezing the mixture; and
(f) washing the frozen mixture and lyophilizing the same to thus produce a sponge including the extracellular matrix of the decellularized kidney tissue.

In the present invention, the “tissue regeneration” means that normal epidermal cells, fibroblasts, etc. grow and proliferate from wound or damaged or deleted tissues to thus recover the wound or damaged or deleted tissues, thereby restoring the functions of tissue.

In the present invention, the “hemostasis” is a process of stopping bleeding, and means that blood within damaged blood vessels are maintained.

In the present invention, the “decellularization” means that cells are removed from body tissues, and an extracellular matrix (ECM) is remained. The tissue in which only the structure of protein is remained, does not occur immune response so that it may be used as a bio-implantable medical device.

In the present invention, the step (a) may be a process of cutting the separated kidney tissue into sections having a thickness of 0.1 to 1 mm, 0.1 to 0.8 mm, 0.1 to 0.6 mm, 0.2 to 1 mm, 0.2 to 0.8 mm, 0.2 to 0.6 mm, 0.3 to 1 mm, 0.3 to 0.8 mm, 0.3 to 0.6 mm, 0.4 to 1 mm, 0.4 to 0.8 mm, or 0.4 to 0.5 mm, and then, washing the sections to thus primarily remove blood or urine component contained in the tissue, wherein the washing may include agitating the sections with distilled water at room temperature 1 to 5 times, 1 to 3 times, 2to 5 times, 2 to 3 times, or 3 times for 10 to 60 minutes, 10 to 40 minutes, 10 to 30 minutes, 20 to 60 minutes, 20 to 40 minutes, 20 to 30 minutes, 30 to 60 minutes, 30 to 40 minutes, or 30 minutes per time, but is not limited thereto.

In the present invention, the step (b) is a process of surfactant treatment and agitation, and may include treating along with sodium chloride, primary agitation at room temperature for 30 minutes to 1 hour 30 minutes, 30 minutes to 1 hour, 50 minutes to 1 hour 30 minutes, 50 minutes to 1 hour, 1 to 1 hour 30 minutes or 1 hour, and secondary agitation for 12 to 20 hours, 12 to 18 hours, 12 to 16 hours, 14 to 20 hours, 14 to 18 hours, 14 to 16 hours or 16 hours, so as to remove blood residue and urine component in the tissue, and other components including a nucleus within a cell membrane of the tissue, but is not limited thereto. At this time, the surfactant may be Triton X-100 at a concentration of 0.5 to 2%, 0.5 to 1.5%, 0.5 to 1%, 1 to 2%, 1 to 1.5% , or 1%, but is not limited thereto. Further, a concentration of sodium chloride may range from 0.01 to 1 M, 0.01 to 0.5 M, 0.01 to 0.1M, 0.05 to 1 M, 0.05 to 0.5 M, 0.05 to 0.1 M, 0.1 to 1 M, 0.1 to 0.5 M, or 0.1 M, but is not limited thereto.

In the present invention, the step (c) may include: after removing all of the surfactant and sodium chloride solution (a hypertonic solution), washing the tissue sections with distilled water at room temperature 1 to 5 times, 1 to 3 times, 2 to 5 times, 2 to 3 times, or 3 times for 5 to 20 minutes, 5 to 15 minutes, 5 to 10 minutes, 10 to 20 minutes, 10 to 15minutes, or 10 minutes per each time, so as to remove tissue/cell residue remaining in the tissue and the hypertonic solution residue; dissolving DNase in a phosphate buffer solution at a concentration of 10 to 100 U/ml, 10 to 70 U/ml, 10 to 50 U/ml, 30 to 100 U/ml, 30 to 70 U/ml, 30 to 50 U/ml, 50 to 100 U/ml, 50 to 70 U/ml, or 50 U/ml to prepare a treatment solution, then, placing the kidney tissue sections therein and agitating the solution at 32 to 40° C., 32 to 39° C., 32 to 38° C., 32 to 37° C., 35 to 39° C., 35 to 38° C., 35 to 37° C., or 37° C. for 3 to 8 hours, 3 to 7 hours, 3 to 6 hours, 5 to 8 hours, 5 to 7 hours, 5 to 6 hours, or 6 hours, so as to remove residual DNA component in the kidney tissue, but is not limited thereto.

In the present invention, between the steps (c) and (d), a step of washing the tissue sections with distilled water at room temperature 1 to 5 times, 1 to 3 times, 2 to 5 times, 2to 3 times, or 3 times for 5 to 15 minutes, 5 to 10 minutes, 10 to 15 minutes, or 10 minutes per each time; preparing a mixture solution of peracetic acid at 0.01 to 1%, 0.01 to 0.05%, 0.01 to 0.1%, 0.05 to 1%, 0.05 to 0.5%, 0.05 to 0.1%, 0.1 to 1%, 0.1 to 0.5%, or 0.1%, or ethanol at 1 to 6%, 1 to 5%, 1 to 4%, 3 to 6%, 3 to 5%, 3 to 4%, or 4%, and then, agitating the mixture solution at room temperature for 10 to 30 minutes, 10 to 25 minutes, 10 to 20minutes, 15 to 30 minutes, 15 to 25 minutes, 15 to 20 minutes, or 20 minutes to sterilize the solution; and, after agitation, washing the product with a phosphate buffer solution 1 to 5times, 1 to 3 times, 2 to 5 times, 2 to 3 times, or 3 times in order to remove the mixture solution and other residues may be further included, but is not limited thereto.

In the present invention, the step (d) may be a process of lyophilizing the kidney tissue free of DNA component to remove moisture, forming the lyophilized tissue into a dried protein form, and then, keeping the product frozen, so as to obtain an extracellular matrix of decellularized kidney tissue, but is not limited thereto.

In the present invention, the step (e) may be a process of mixing the extracellular matrix of the decellularized kidney tissue (kdECM) with a cross-linking agent, followed by agitation and freezing to thus produce cryogel, wherein the kdECM may be prepared in a liquid form by agitating kdECM in 0.2 to 1 M, 0.1 to 0.7 M, 0.1 to 0.5 M, 0.3 to 0.7 M, 0.3to 0.5 M or 0.5 M acetic acid and powdery pepsin for 3 to 6 days or 4 to 5 days, but is not limited thereto. The pepsin may be used in 0.05 to 0.2 times, 0.05 to 0.1 times, 0.1 to 0.2times, or 0.1 times relative to dried kdECM mass, but is not limited thereto.

In the present invention, the cross-linking agent may be one or more selected from the group consisting of 1-ethyl-3-(3-dimethylaminopropyl) carbodiimide hydrochloride (EDC) and N-hydroxysuccinimide (NHS), but is not limited thereto. In this case, as a solvent for preparing the EDC and NHC solutions, 0.5 to 0.7 M, 0.5 to 0.65 M, 0.6 to 0.7M, 0.6 to 0.65 M, or 0.625 M MES buffer solution (4-morpholine ethane sulfonic acid monohydrate) may be used and pH value thereof may be adjusted to pH 5 to 7, 5 to 6, 6 to 7, or 6, but is not limited thereto.

In the present invention, the cross-linking agent used herein may be admixed in EDC solution:NHS solution—2 to 6:1, 2 to 5:1, 2 to 4:1, 3 to 6:1, 3 to 5:1, 3 to 4:1, 4 to 6:1, 4 to 5:1, or 4:1 mole ratio, but is not limited thereto.

In the present invention, the cross-linking agent used herein may be admixed in EDC solution: NHS solution=1:0.5 to 2, 1:0.5 to 1, 1:1 to 2, 1:1 to 1.5, or 1:1 mass ratio, but is not limited thereto.

In the present invention, in the step (e), a solution of the extracellular matrix of decellularized kidney tissue: a cross-linking agent solution mixed with EDC solution and NHS solution=4 to 8:1, 4 to 7:1, 4 to 6:1, 5 to 8:1, 5 to 7:1. 5 to 6:1, 6 to 8:1, 6 to 7:1or 6:1 mass ratio, and agitated, but is not limited thereto.

In the present invention, the freezing in the step (e) may be performed at −40 to −10° C., −40 to −20° C., −30 to −10° C., −30 to −20° C., −20 to −10° C., or −20° C. for 12 to 36 hours, 12 to 30 hours, 12 to 24 hours, 18 to 36 hours, 18 to 30 hours, 18 to 24 hours, or 24 hours, but is not limited thereto.

In the present invention, the step (f) may be a process of washing the frozen cryogel with distilled water at room temperature to remove EDC/NHS solution and residue after cross-linking, and then, lyophilizing the remaining product for 1 to 5 days, 1 to 3 days, 2 to 5 days, 2 to 3 days, or 3 days, so as to produce a porous material including an extracellular matrix of decellularized kidney tissue, but is not limited thereto.

Further, the present invention provides a porous material for tissue regeneration and hemostasis, comprising an extracellular matrix of decellularized kidney tissue,
wherein the extracellular matrix of decellularized kidney tissue has a structure formed of chemical cross-linking bonds between proteins.

In the present invention, the “cross-linkage” is a chemical bond to connect adjacent two atoms in one or more polymer molecule, which may include a hydrogen bond, an ionic bond, or a covalent bond. According to one embodiment of the present invention, the cross-linkage may be an amide bond, but is not limited thereto.

In the present invention, the chemical cross-linking bond may be produced by one or more cross-linking agent selected from the group consisting of 1-ethyl-3-(3-dimethylaminopropyl) carbodiimide hydrochloride (EDC) and N-hydroxysucinimide (NHS), but is not limited thereto.

In the present invention, when using the above EDC and NHS as the cross-linking agent, a chemical cross-linking process may proceed as shown in the following scheme 1.

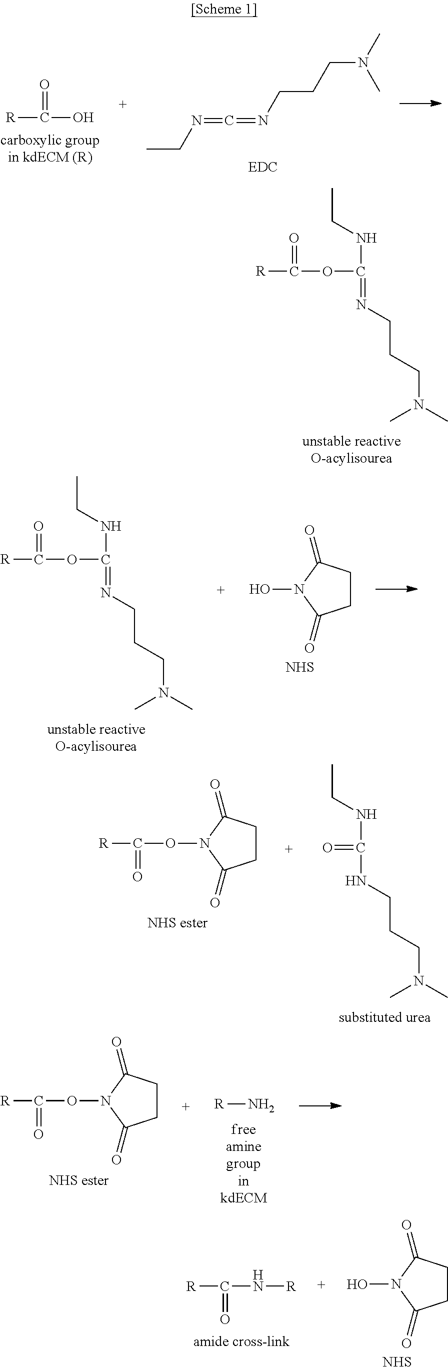

In the present invention, the porous material may have one or more among the following activities, but is not limited thereto:
(i) reduction of a degradation rate;
(ii) hemostasis (bleeding stop) in damaged site of tissuc;
(iii) tissue regeneration; and
(iv) increase in proportion of M2 macrophages.

In another embodiment of the present invention, the porous material may be used for transplantation in vivo or as an external skin preparation, but is not limited thereto.

Further, the present invention provides a composition for tissue regeneration and hemostasis, comprising the porous material for tissue regeneration and hemostasis.

In this regard, the composition may be a pharmaceutical composition, but is not limited thereto.

Further, the present invention provides a kit for tissue regeneration and hemostasis, including the porous material for tissue regeneration and hemostasis.

In the present invention, the “kit” means a tool or device for tissue regeneration and hemostasis, which includes the porous material for tissue regeneration and hemostasis. The kit of the present invention may include other components, composition, solution, device, etc. commonly required for a method of tissue regeneration and hemostasis as well as the porous material described above. In this case, the porous material for tissue regeneration and hemostasis may be applicable once or more without limitation of the number of use and, in particular, there is no limitation before and after application of each material. Further, separate materials may be concurrently or separately applicable.

In the present invention, the kit may include: a container; an instruction (or manual); and a porous material for tissue regeneration and hemostasis. The container may play a role of packaging the porous material, or for storage or fixation thereof. The container may have different forms such as bottle, tub, sachet, envelope, tube, ampoule, etc., which may be partially or entirely formed using plastic, glass, paper, foil, wax, etc. The container may be provided with a cap or stopper, which is originally a part of the container or can be attached to the container by any mechanical, adhesive or other means, therefore, completely or partially separable from the container. The kit may further include an external package, and the external package may include an instruction for use of components enclosed therein.

Further, the present invention provides a pharmaceutical composition for prevention or treatment of diseases caused by tissue damage, comprising the porous material for tissue regeneration and hemostasis as an active ingredient.

According to the present invention, the disease is not particularly limited so far as it is any disease due to damage in one or more tissues selected from the group consisting of kidney tissue, liver tissue, heart tissue, intestinal tissue, skin tissue, stomach tissue, pancreas tissue, brain tissue, thyroid tissue, lung tissue, and spleen tissue. For example, traumatic kidney damage, nephrolith, renal failure, polycystic kidney disease, renal cancer, traumatic liver damage, liver failure, hepatitis, hepatic cirrhosis, liver cancer, traumatic heart damage, heart failure, cardiac infarction, angina, arrhythmia, myocarditis, cardiomyopathy, heart cancer, traumatic intestinal damage, ulcerative colitis, Crohn's disease, colon cancer, traumatic skin damage, dermatitis, skin cancer, traumatic stomach damage, gastritis, gastric ulcer, stomach cancer, traumatic pancreatic damage, pancreatitis, pancreatic cancer, traumatic brain damage, cerebral stroke, degenerative brain diseases, cerebral infarction, traumatic thyroidal damage, thyroidal nodule, thyroidal cancer, thyroiditis, traumatic lung damage, pnumoneia, tuberculosis, lung cancer, idiopathic pulmonary fibrosis, lung failure, traumatic spleen damage, spleen rupture, or splenomegaly, and the like, but is not limited thereto.

In the present invention, the composition may be used for: treatment of tissue damaged site; or tissue regeneration as well as hemostasis when the tissue is cut or under incision operation, however, is not limited thereto.

The pharmaceutical composition of the present invention may further include desirable carriers, excipients and diluents. For example, the excipient may be one or more selected from the group consisting of diluents, binders, disintegrating agents, lubricants, adsorbants, humectants, film-coating materials, and controlled release-additives.

The pharmaceutical composition of the present invention may be used by formulating a sponge containing an extracellular matrix of decellularized kidney tissues into the form of gauze, sheet, patch, pad, etc. according to any conventional method.

The carrier, excipient and diluent possibly contained in the pharmaceutical composition according to the present invention may include, for example, lactose, dextrose, sucrose, oligosaccharide, sorbitol, mannitol, xylitol, erythritol, maltitol, starch, acacia gum, alginate, gelatin, calcium phosphate, calcium silicate, cellulose, methyl cellulose, microfine cellulose, polyvinyl pyrolidone, water, methylhydroxybenzoate, propylhydroxybenzoate, talc, magnesium stearate and mineral oil.

The formulation may be produced using diluents or excipients such as fillers, extenders, binders, wetting agents, disintegrating agents, surfactants, etc. generally used in the art.

The pharmaceutical composition of the present invention may be administered in a pharmaceutically effective amount. In the present invention, the “pharmaceutically effective amount” means an amount sufficient to treat a disease with a rational benefit/damage rate applicable to medical treatment, wherein an effective dose level may be determined on the basis of some factors including, for example, types of disease of a patient, severity, activity of drug, sensitivity to drug, administration time, administration route and discharge rate, treatment period, another drug used simultaneously, as well as other factors well known in the medical field.

The pharmaceutical composition according to the present invention may be administered as a separate medicine or in combination with other remedy drugs, may be administered with conventional therapeutics in sequential order or simultaneously, and may be administered in single or multiple doses. In consideration of the above factors, it is important to administer a minimum amount of the composition that can obtain maximum effects without side effect. This may be easily determined by those skilled in the art to which the present invention pertains.

The pharmaceutical composition of the present invention may be directly treated or transplanted on a tissue damaged site or a surgical site of an individual, and the pharmaceutical composition of the present invention may be determined according to types of drug as the active ingredient as well as various relevant parameters such as a disease to be treated, administration route, age, gender and weight of a patient, and severity of disease, etc

Further, the present invention provides a method for prevention or treatment of diseases caused by tissue damage, comprising treating a subject in need of a composition comprising the porous material for tissue regeneration and hemostasis as an active ingredient, with the composition.

Further, the present invention provides a use of a composition comprising the porous material for tissue regeneration and hemostasis as an active ingredient, the use comprising prevention or treatment of diseases caused by tissue damage.

Still further, the present invention provides a use of the porous material for tissue regeneration and hemostasis, the use comprising production of a preparation for prevention or treatment of diseases caused by tissue damage.

In the present invention, the “individual” means a subject in need of treatment of a disease, more particularly, includes human or non-human primates, and mammals such as mouse, rat, dog, cat, horse and cow.

In the present invention, the “treatment” means providing the composition of the present invention to an individual by any suitable method.

In the present invention, the “prevention” means all actions for inhibiting or delaying occurrence of target diseases, while the “medical treatment” means all actions for improving or beneficially altering a target disease and metabolic abnormal symptoms thereof by administering the pharmaceutical composition of the present invention.

Hereinafter, preferred embodiments will be proposed to aid understanding of the present invention. However, these examples are only provided to more easily understand the present invention, and the present invention is not limited thereby.

### EXAMPLE

### Example 1. Fabrication of a Sponge of Extracellular Matrix of Decellularized Kidney Tissue With Chemical Cross-Linked Amide Bond

A fresh state of a kidney of a pig with a health certificate from the veterinary medicine college of Chungbuk University was secured, and surrounding fat tissues and kidney capsule were removed, followed by storage at −80° C. till decellularization proceeds. The decellularization was conducted by the following processes.

The frozen kidney was cut into sections with a thickness of 0.4 to 0.6 mm, placed in a beaker, followed by agitating and washing with third-distilled water at room temperature three times for 30 minutes per each time so as to primarily eliminate blood and urine component contained in the tissue. The distilled water was completely removed and replaced with a hypertonic solution prepared of 0.1M sodium chloride (Samchun) and 1% concentration of Trion X-100 as a surfactant, then, the solution was firstly agitated at room temperature for lhours and secondly agitated for 16 hours to remove blood and urine component remaining in the tissue, as well as components including a nucleus of a cell membrane of the tissue. After completely removing the hypertonic solution, the tissue section was washed with third-distilled water at room temperature three times for 10 minutes per each time in order to remove tissue/cell residue and hypertonic solution residue. After then, a phosphate buffer solution (10X PBS; Genetch) was prepared at 1% concentration, and DNase was dissolved therein and prepared with a concentration of 50 U/ml. In a beaker including the above prepared solution, the tissue section was added and then agitated at 37° C. for 6 hours. Through this, DNA component possibly remaining in a cell nucleus of the tissue was eliminated. Then, in order to wash the remaining component, the washing was carried out with third-distilled water at room temperature three times for 10 minutes per each time. In a sterilization step, a mixed solution of 0.1% peracetic acid (Merk) and 4% ethanol (Merk) was prepared and added to the above solution, followed by agitating at room temperature for 20 minutes to proceed sterilization. After agitation, in order to the mixed solution and other residue, the above solution was washed with 1x PBS at room temperature three times. The completely washed kidney tissue section was finally subjected to lyophilization to remove moisture, prepared into a dry protein form, followed by keeping the same in a freezer.

After the decellularization process according to the above procedure, kidney decellularized extracellular matrix (kdECM) was obtained and used to produce kidney derived extracellular matrix sponge (kdES). The overall production process includes: mixing complex materials→freezing at −20° C.→washing at room temperature→lyophilization.

The complex materials may include kdECM solution, EDC solution, and NHS solution. Firstly, in order to prepare kdECM in a liquid state from the original dry state, kdECM was agitated in 0.5 M acetic acid (Merk) an powdery pepsin (Merk) for 4 to 5 days using a magnet and a stirrer. Pepsin was used in an amount of 1/10 mass of dried kdECM, a final solution concentration of kdECM was progressed to 3 w/v %. For example, in order to prepare 10 ml of kdECM solution, 300 mg of dried kdECM was agitated along with 30mg of pepsin.

In order to prepare EDC (1-ethyl-3-(3-dimethylaminopropyl) carbodiimde hydrochloride; ThermoFisher Scientific) solution and NHS (N-Hydroxysuccinimide; Merk) solution, 0.625 M MES buffer solution (4-morpholine ethane sulfonic acid monohydrate; Merk) as a solvent was prepared, and acidity was adjusted with sodium hydroxide (NaOH; Biosesang) to pH 6 at which EDC and NHS are possibly activated.

Each of EDC and NHS was dissolved in MES buffer solution in a mass to reach a final target concentration, the prepared EDC and NHS solutions were admixed in 1:1mass ratio and directly added to kdECM solution while agitating. A mass ratio of kdECM solution: EDC/NHS mixed solution was adjusted to 6:1 for use. At this time, a molar ratio of EDC:NHS was defined to 4:1 for use (12 mM: 3 mM, 24 mM: 6 mM, 48 mM: 12mM).

The mixture with completed agitation was moved to a freezing room at −20° C. within 1 minute, followed by freezing to secure cryogel.

When EDC and NHS are used as a chemical cross-linking agent for production of cryogel, the cross-linking process proceeds according to Scheme 1 below.

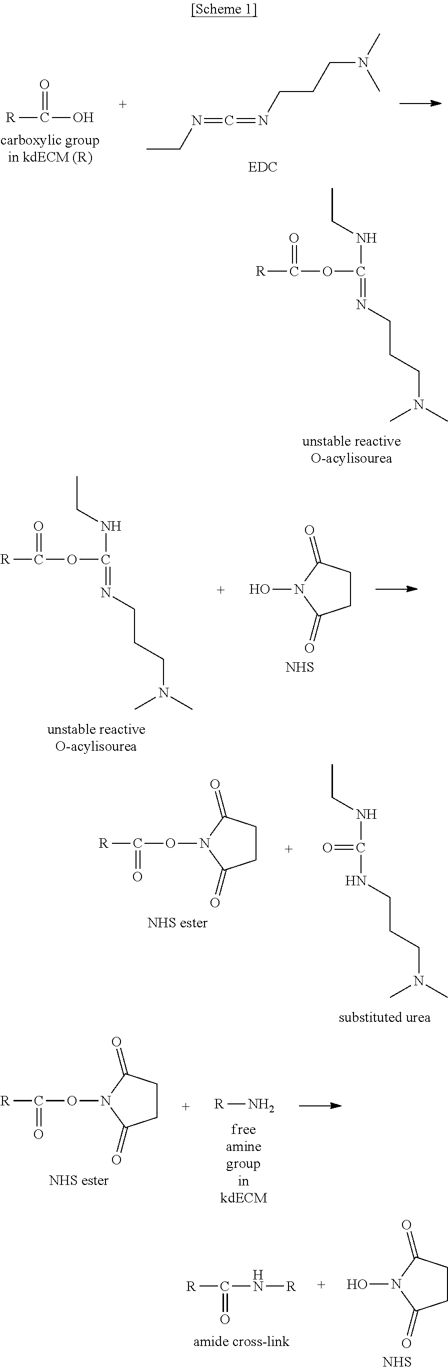

More particularly. EDC is bound to a carboxyl group in a protein chain during freezing to form a reaction-sensitive O-acylisourea compound in unstable state. To a resulting product of a first scheme above. NHS is added and reacted to form more stable NHS ester. This NHS ester is reacted with an amine group in other amino acid contained in the compound to form an amide bond. After the binding reaction, the existing NHS in NHS ester is return to the original state (R: a protein chain contained in kdECM, which is relevant to chemical cross-linkage).

The completely frozen cryogel was used to remove EDC/NHS solution and cross-linking residue by replacing the distilled water with third-distilled water at room temperature. The distilled water was changed over total 24 hours including 15 minutes, 15minutes, 30 minutes, 1 hour, 4 hours, 6 hours and 12 hours after starting the washing process.

The completely washed cryogel was lyophilized for 3 days after removing the distilled water, thereby completing the production of a final kdES.

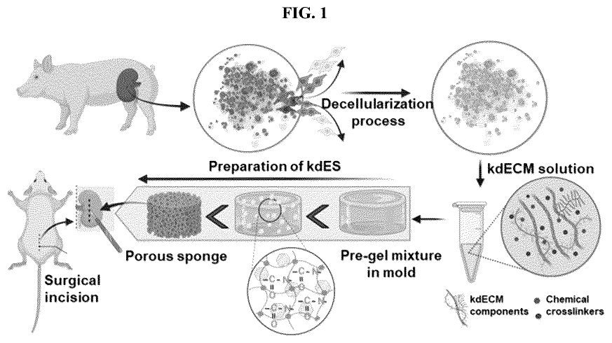

Such a process including the production to application of kdES was schematically illustrated in FIG. 1.

### Example 2. Cross-Linking Analysis and Fourier Transform Infrared Spectroscopy Assay

Using kdES synthesized according to the method in Example 1, cross-linking degree analysis and Fourier Transform Infrared Spectroscopy assay were performed.

More particularly, in order to execute Ninhydrin assay, samples before and after the cross-linking were secured each in 5 mg and heated at 100° C. for 20 minutes while agitating with 2% Ninhydrin. After cooling the heated samples to room temperature, 10 ml of 50% isopropanol was added to each sample, followed by measuring absorbance to a solution at 570 nm wavelength by a spectrophotometer. For amino acid concentration substitution per absorbance range, a standard curve using glycine was secured, and a variation in amount of free amino acid groups per kdES sample was indirectly confirmed.

As the variation of free amino acid before and after the cross-linking bond is increased, it means that the cross-linking bond was largely formed, thereby indicating higher cross-linking bond value.

Further, in order to perform Fourier Transform Infrared Spectroscopy, a transparent sheet containing dried kdES was fabricated. For the fabrication of the transparent sheet, the samples before and after kdES cross-linking were each prepared in 2mg, pulverized along with 10 mg of potassium bromide, placed in a presser to produce a high-contact transparent film, followed by mounting the same in Vertex 70 Infrared spectrophotometer (Bruker) for assay at a wavelength range of 4000 to 500 cm⁻¹.

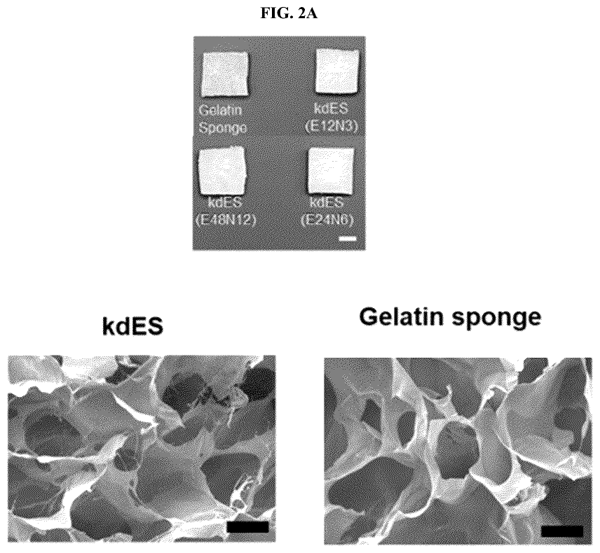

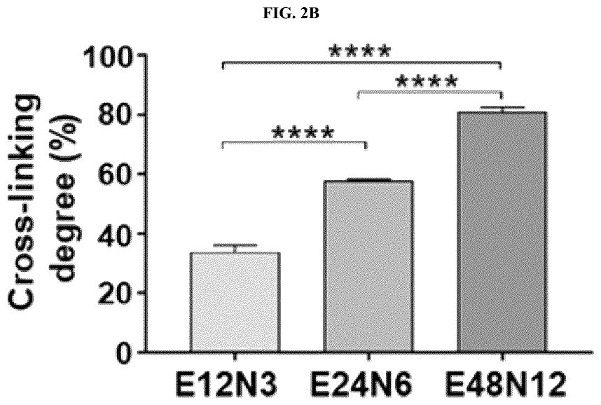

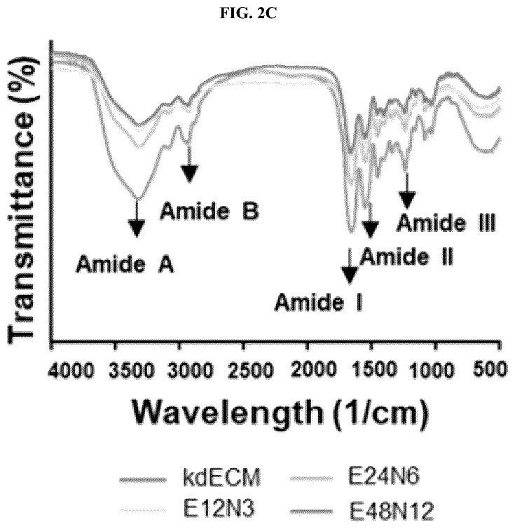

Firstly, the gelatin sponge commercially available in the art and kdES produced at different EDC/NHS concentrations (E12N3, E24N6, and E48N12)were compared in terms of morphology (upper figure, scale bar: 5 mm). Further, microstructures of kdES and the gelatin sponge were monitored through a scanning electron microscope (SEM) and compared (lower figure, scale bar: 100 μm) as shown in FIG. 2A.

The result of confirming cross-linking degrees of E12N3, E24N6, and E48N12 ) as shown in FIG. 2B, while the result of analyzing infrared transmittance of kdECM, E12N3, E24N6, and E48N12 through Fourier Transform Infrared Spectroscopy was shown in FIG. 2C (‘E number N number’ denote all kdES, indicating the number based on the concentration of used EDC/NHC (cross-linking agent). For instance, when kdES is produced using EDC/NHS at 12 mM/3 mM, it is represented by E12N3).

As shown in FIG. 2B, the measured values obtained through Ninhydrin assay were numerically converted into the cross-linking degree and it was confirmed that, as the concentration of EDC/NHS solution used for production of kdES increases, the cross-linking degree is also increased.

Further, as shown in FIG. 2C, as a result of observing peaks at amide A, amide B, amide I, amide II and amide III bands, which were generated due to oscillation of a peptide bond to form collagen, it was confirmed that a peak area was sharper with increased EDC/NHS concentration while infrared transmittance was lowered.

### Example 3. Analysis of Swelling Rate and Swelling Dynamics

Using kdES (E12N3, E24N6, and E48N12)synthesized according to the method in Example 1 as well as the gelatin sponge, swelling rate and swelling dynamics were analyzed.

More particularly, after measuring a mass of each of porous material samples in dry state (kdES and the gelatin sponge), the sample was immersed in distilled water to determine a variation in mass. The mass over time was measured and the measurement was conducted until the sample mass becomes equilibrium state (that is, it becomes a saturated state without further mass change). The swelling rate was calculated by way of dividing a mass of the sample in equilibrium state with distilled water by a dry sample mass in a dried sate before initial immersion.

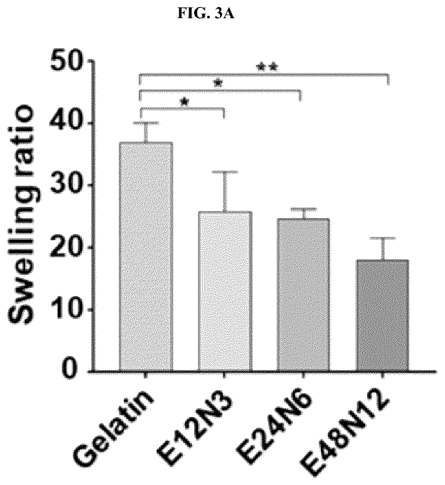

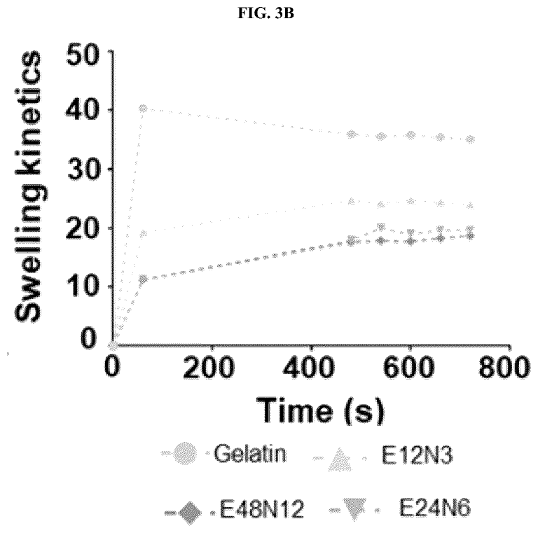

As shown in FIGS. 3A and 3B, as a result of analyzing the swelling rate and the swelling dynamics of each 10 mg sponge sample in distilled water, an absorption ability of the gelatin sponge was measured to be higher than that of kdES samples and, among kdES samples, and it was confirmed that, as a concentration of the cross-linking agent is higher, the absorption rate tends to be decreased.

### Example 4. Biodegradation Experiment

In ex vivo environment (before transplantation to an animal, an imitation environment thereof), a difference in degradation rates of kdES (E12N3, E24N6, and E48N12) and the gelatin sponge was confirmed through preliminary biodegradation experiments.

More particularly, each porous material sample was cut in a dimension of 4 mm ×4 mm×2 mm, placed in a 48-well dish and firstly immersed in PBS solution completely. The completely immersed samples were moved to 58-well dishes, each including 200 μL collagenase III solution after completely removing surface moisture, followed by starting biodegradation experiment. A biodegradation rate was measured according to the following formula:

$$Biodegradation rate ( \% ) = [ ( {W}_{0} - {W}_{t} ) / {W}_{0} ] \times 100$$

wherein Wₒ refers to a weight of porous material in dry state, Wₜ indicates weight per time.

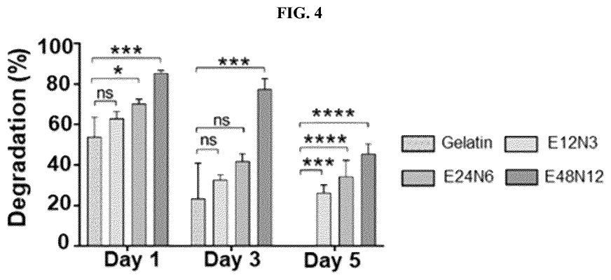

Consequently, as shown in FIG. 4, the gelatin sponge has residual mass of 50% on day 1 and 20% on day 3, compared to the starting of experiment, and has been completely decomposed on day 5. On the other hand, kdES group showed more delayed degradation, and it was confirmed that the degradation is delayed with increased concentration of EDC/NHS cross-linking agent.

### Example 5. Cell Viability and Growth Rate Assay

In ex vivo environment, how much kdES (E12N3, E24N6, and E48N12) synthesized according to the method of Example 1 is a bio-compatible substance (whether or not a material on which cells are well alive and desirably grown (or proliferated)) was confirmed using kidney fibroblasts as compared to the gelatin sponge.

More particularly, before cell seeding, both of the gelatin sponge (control group) and the kdES were cut in a size of 4 mm×4 mm×2 mm, followed by sterilization through UV light. Thereafter, the section was plated on 24-well dish including Dulbecco's Modified Eagle culture medium supplemented with 10% fetal bovine serum; FBS) and 1% penicillin/streptomycin. Then, 1 ml of cell suspension was added at a concentration of 1×106 cells/mL to each well containing the sections. All cells were incubated in saturated atmosphere of 5% CO at 37° C., and the medium was replaced every 48 hours.

The viability of kidney fibroblasts was assessed according to LIVE/DEAD® assay. Firstly, a cell seeded sample was moved to a cofocal microscopic dish, the sample was washed and stained with 4 mL of sterile PBS containing 4 μL of 2 μM calcein AM and 8 μL of 4 μM ethidium homodimer I (EthD-I), followed by incubating the sample in a dark room for 30 minutes. After washing with PBS, the sample was observed by the cofocal microscope. The secured shape was used to calculate the viability of each sample through ImageJ program.

Further, in order to determine a cell growth rate, CCK-8 method was used. More particularly, CCK-8 solution is admixed with the medium in 1:20 ratio and is introduced into each well containing a porous material sample as well as cells, followed by incubation for 1 hour. Then, 100 μL of supernatant in each well was moved to 96-well, followed by measuring absorbance at 450 nm wavelength and calculating a relative cell growth rate with reference to the measured value.

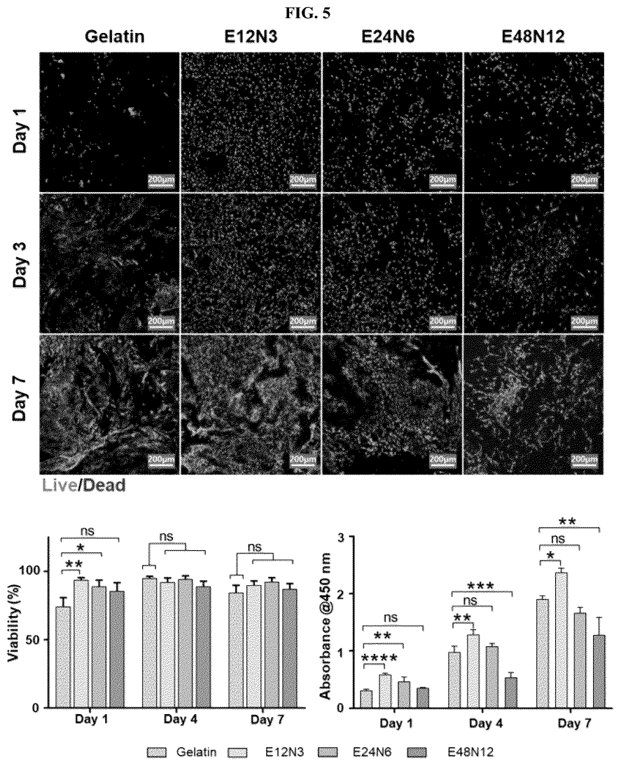

Consequently, as shown in FIG. 5, the cell viability was relatively low in the gelatin sponge on day 1 after cell seeding, however, in view of other observations, all maintained more than 90% and thus showed no significant difference (scale bar: 200 um). Further, as a result of confirming the cell viability with the measured absorbance, there was a significant difference between kdES and, among them, E12N3 at the lowest concentration of the cross-linking agent exhibited the highest cell growth rate (*p<0.05, **p<0.01,***p<0.001, ****p<0.0001, ns: not significant).

### Example 6. Ex Vivo Blood Compatibility Assessment

Using kdES (E12N3, E24N6, and E48N12)synthesized according to the method in Example 1 as well as the gelatin sponge, ex vivo blood compatibility as one of items used for assessment of hemostats was evaluated. Further, it was determined whether cells in the blood are lysed (destructed) or not when the blood is treated with kdES.

More particularly, the blood compatibility of the porous material was evaluated by hemolysis assay. 100 μl of fresh blood taken from 8 weeks-old SD rat under euthanasia was prepared in each 15 mL tube, then, the gelatin sponge or kdES section was prepared in a dimension of 5 mm×5 mm×2 mm in each tube. Then, after mixing the prepared section for 1 minute using voltex, 5 mL of PBS solution was added to the tube, followed by uniformly pipetting. 15 mL tube including the agitated sample was treated in a centrifuge at 3000 rpm for 5 minutes and then a supernatant was secured. 100 μl of supernatant in each group was moved to 96-well dish, followed by measuring absorbance at 540 nm in a micro-plate reader. The control group was treated with PBS containing 0.1% Triton X-100to measure absorbance. Lysis rate of the porous material section was calculated by the following equation.

$$Hemolysis rate ( \% ) = ( {A}_{s} / {A}_{t} ) \times 100$$

wherein Aₛ refers to an absorbance value obtained in each sponge sample while Aₜ indicates an absorbance value obtained in Triton X-100 group.

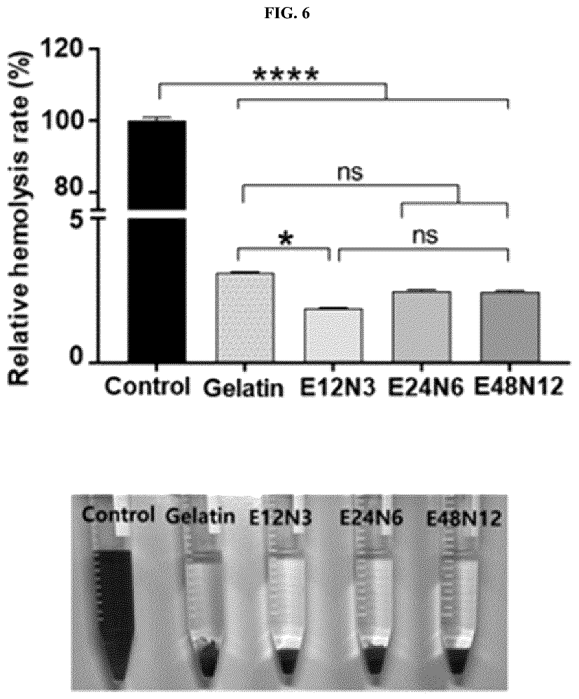

Consequently, as shown in FIG. 6, in the case where blood was treated on the gelatin sponge, the cell lysis rate was higher than that of kdES group. Further, it was confirmed that, among kdES, E12N3 group has the lowest cell lysis (*p<0.05,****p<0.0001, ns: not significant).

### Example 7. Assessment of Ex Vivo Hemostasis Ability

Animal blood was taken and blood only was placed in an empty space of a well plate. Or, kdES (E12N3) synthesized according to the method of Example 1 as well as the gelatin sponge was treated with the blood, followed by measuring/evaluating blood coagulation extents over time (30 seconds, 1 minutes, 2 minutes, 3 minutes, 4 minutes and 5 minutes) (when ‘kdES’ only is expressed in the present invention, it means kdES representative group, that is, E12N3 group which was confirmed as predominant conditions in the above examples).

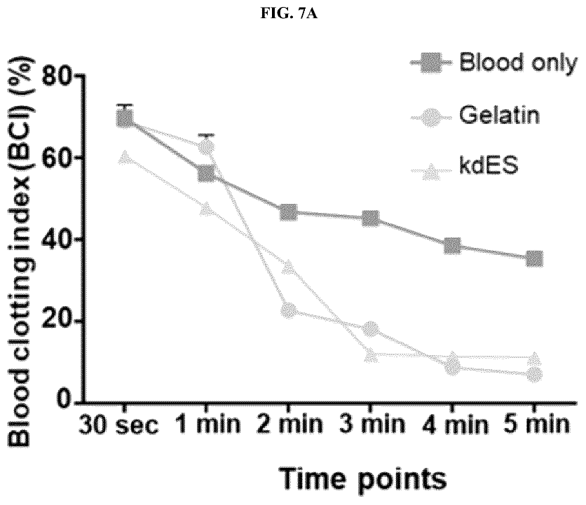

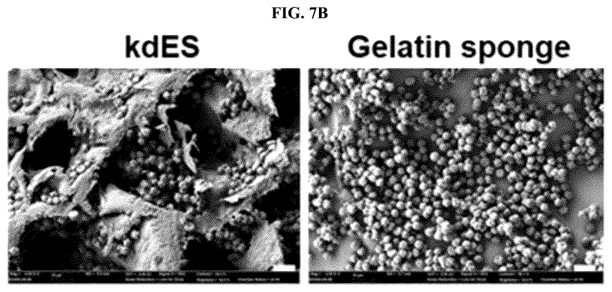

Consequently, as shown in FIG. 7A, when blood only was treated, natural coagulation was induced but has relatively low speed. In fact, when bleeding is continued, hemostasis occurs with non-significant extent, while the gelatin sponge and kdES showed rapidly high hemostasis effects in 5 minutes.

On the other hand, after adhesion of whole blood cells, SEM images of the gelatin sponge and kdES are shown in FIG. 7B.

### Example 8. Analysis of Functional Enrichment of kdES

Based on data obtained by analyzing total protein configuration of specific materials, kdES synthesized according to the method of Example 1 was subjected to proteomic assay and functional proteins were categorized in terms of physiological functions.

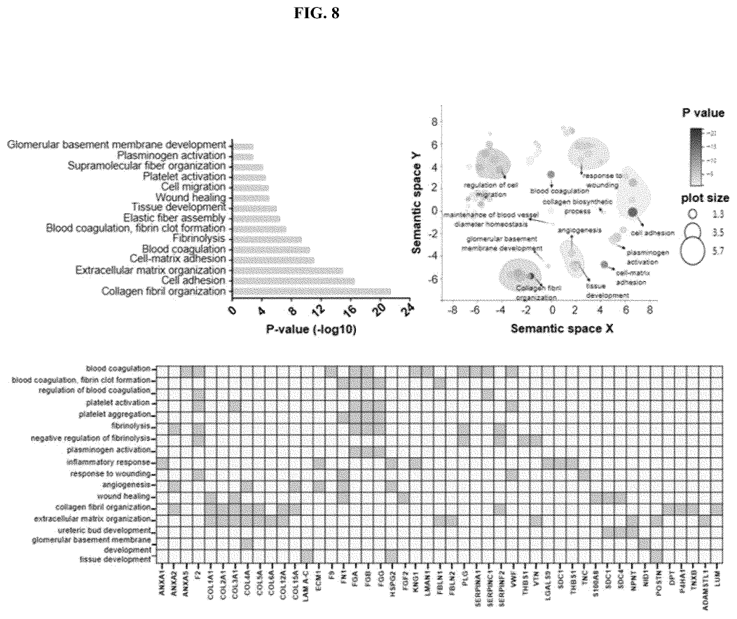

The upper left figure in FIG. 8 illustrates the result of gene ontology (GO) concentration assay of concentrated protein in kdECM after decellularization, which was allocated to top 15 biological process phraseologies assigned relative to p-values, while the upper right figure in FIG. 8 illustrates a semantic plot for biological phraseology (BP) improved in kdECM core matrix. REVIGO (http://revigo.irb.hr/) was used for semantic similarity of BP terms and a position in the chart reflects the same. P-value is displayed with color coding while the size of a circle indicates the frequency of GO terms. As the circle is larger, the more general term is used. Further, the lower figure in FIG. 8 is a chart illustrating a correlation between the protein of kdECM and the top biological processes.

As shown in FIG. 8, according to the proteomic assay of kdES, among functions

of various kinds of proteins contained in kdES, several functions relevant to hemostatic ability and tissue regeneration have been confirmed.

Example 9. Assessment of In vivo Hemostatic Ability

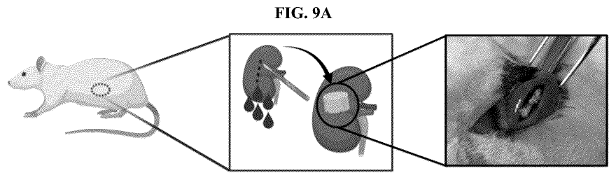

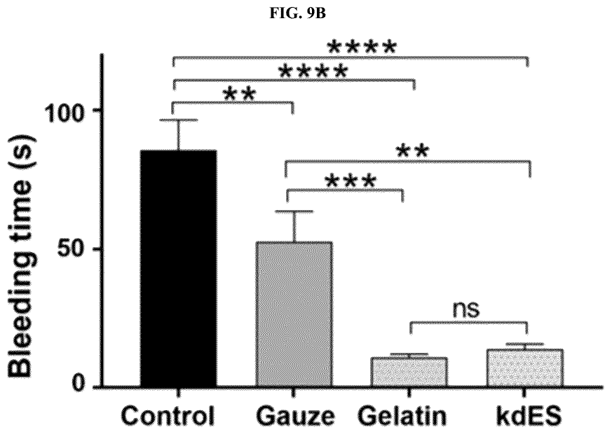

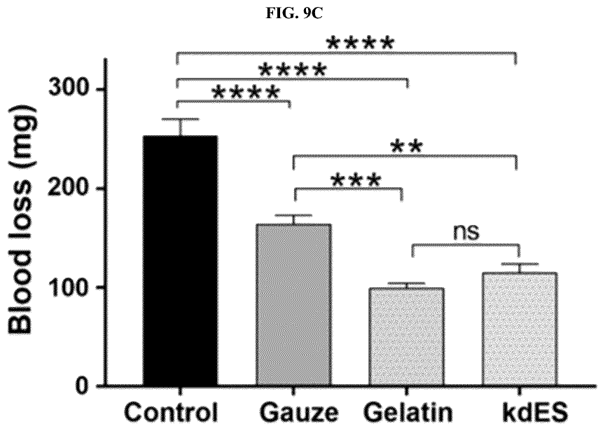

The lateral side of the kidney of 7 to 8-month old rat having a weight of 320 to 390 g was subjected to incision with 1 cm length×1 cm depth to prepare a kidney damage model. Further, kdES synthesized according to the method of Example 1 and the gelatin sponge were transplanted, respectively, followed by hemostasis experiments. As soon as the incision was done, transplantation was immediately conducted. The sponge was fabricated with a square shape of the sponge of 8 mm×8 mm and a thickness of 2 mm, and then, transplanted. As a control group, a rat without any treatment to the cut kidney was used. Further, depending on a bleeding amount (blood loss), a gauze may be applied and thus include in the hemostatic items. FIG. 9A is a schematic view illustrating kidney incision and sponge application.

As shown in FIG. 9B, the hemostatic time was evaluated and, as a result, applying a gauze showed desirable hemostatic effects compared to the control group with simple incision. However, it was confirmed that, when using the gelatin sponge and kdES, the hemostatic time was considerably reduced (**p<0.01, ***p<0.001, ****p<0.000, ns: not significant).

Further, as shown in FIG. 9C, the blood loss was evaluated and, as a result, applying a gauze showed desirable hemostatic effects compared to the control group with simple incision. However, it was confirmed that, when using the gelatin sponge and kdES, the blood loss was considerably reduced (**p<0.01, ***p<0.001, ****p<0.0001, ns: not significant).

### Example 10. Assessment of Kidney Tissue Damage Extent

To the same animal model as in Example 9, the gelatin sponge (GS) or kdES was transplanted and conditions thereof on day 3 and day 28 were histologically assessed.

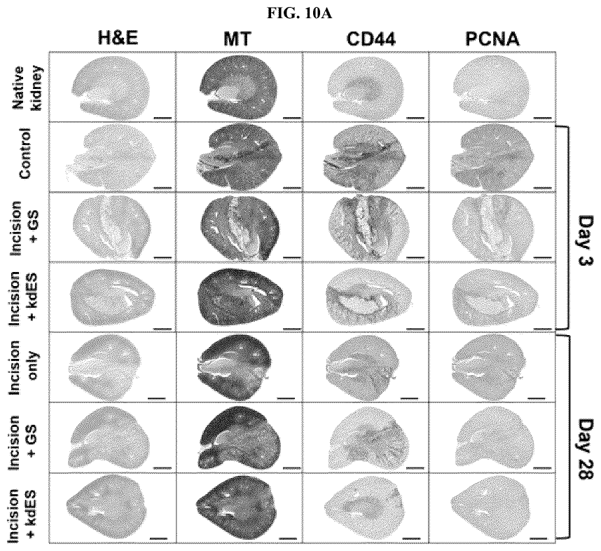

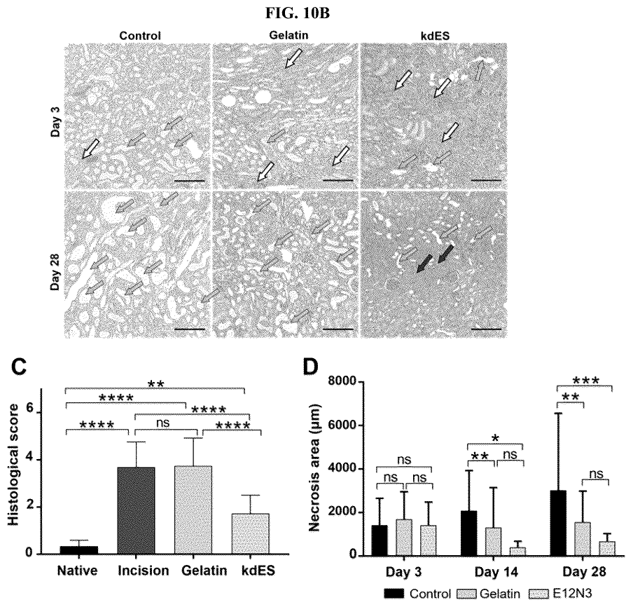

FIG. 10A illustrates the result of investigating wound restoration in the control group (with only incision), the gelatin sponge transplantation group and the kdES transplantation group, respectively, according to histomorphological examination by way of hematoxylin and eosin (H&E), Masson's trichrome (MT), CD44, and proliferating cell nuclear antigen (PCNA) staining on day 3 and day 28 after transplantation of the gelatin sponge (GS) or kdES (scale bar: 200 μm).

FIG. 10B illustrates the results of enlarged H&E staining (upper in FIG. 10B, scale bar: 200 μm) and H&E staining of the control group, the gelatin and kdES groups on day 3 and day 28 after transplantation of the gelatin sponge (GS) or kdES; and quantitative results of tubular necrosis areas in H&E staining histological sections on day 3, day 14 and day 28 after transplantation (lower in FIG. 10B) (*p<0.05, **p<0.01, ***p<0.001,****p<0.0001, ns: not significant).

As shown in FIGS. 10A and 10B, a size of renal tubule was observed and, as a result, it was observed that all of these three groups have occurrence of some initial damage on day 3, however, the gelatin sponge-transplanted group showed worse damage with the passage of 28 days and the control group has further severe level of damage. On the contrary, the kdES-transplanted group did not have worse damage around the kdES transplanted site, and further demonstrated appearance of tissue restoration.

### Example 11. Macrophage Polarization Experiment

A variety of macrophages get involved in wounded tissues, in particular, M1 macrophage (antibody: CD86) as a representative one causes inflammation and expands blood to increase a blood flow rate, while M2 macrophage (antibody: CD206) gets involved in tissue regeneration and restoration. The macrophage may be altered to M1 causing inflammation depending on in vivo environments or M2 promoting reproduction. Accordingly, the macrophage was cultured in ex vivo experiments, seeded on kdES and the gelatin sponge, respectively. Then, after 24 hours and 72 hours, the distribution of M1 and M2 macrophages was confirmed by flow cytometry (FACS) experiments.

More particularly, mouse macrophage cell-line (RAW 264.7) was incubated on 6-well cell culture dish along with a complete medium (Dulbecco's Modified egale's Medium+10% FBS+1% P/S) until the cells reach 60 to 70% confluency. Then, the cells were subjected to sub-culture for 24 hours using a medium including 50 ng/ml concentration of interferon gamma and 10 ng/ml of lipopolysaccharide, thereby inducing phenotype of M1 macrophage. Further, in order to induce M2 cells, the cells were exposed to a medium including 100 ng/ml of IL-10 for 24 hours. After harvesting M1-inducedd cells, the cells were seeded on the gelatin sponge or kdES sections with a cross-section of 4mm×4 mm×2 mm, the section including the cells was replaced with a complete medium, followed by further culturing for 24 hours or 72 hours to thus secure a cell sample. Thereafter, among cells prepared to perform FACS (fluorescence activated cell sorting) assay, 1×10⁶ cells were collected and treated with CD86/FITC antibody and CD206/APC antibody, respectively, at 4° C. for 30 minutes and 1 hour, followed by performing flow cytometry measurement assay. The data were analyzed by Flowjo Analysis Software.

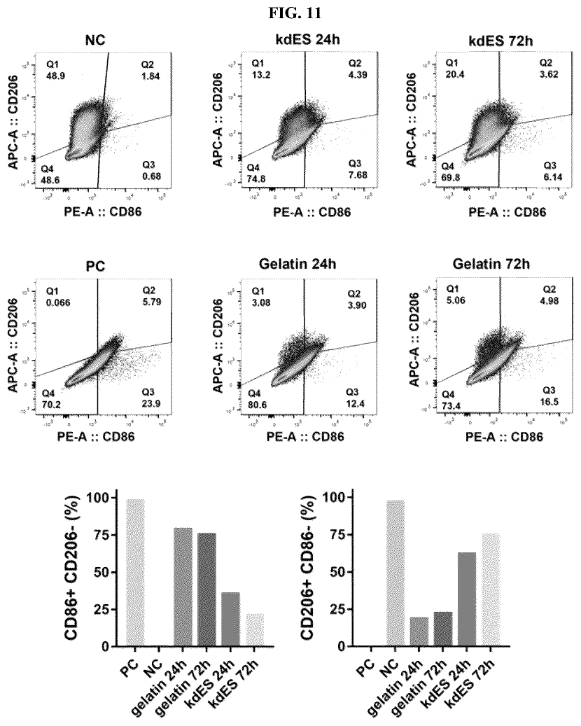

From the result of flow cytometry assay shown in the upper figure in FIG. 11, cell areas (Q1, Q3), in which only one of CD206 or CD86 was expressed (CD206+ or CD86+), are selected among four (4) quadrants, and M1 or M2 rate graph is prepared. Consequently, as shown in the lower figure in FIG. 11, when the cells were seeded on the gelatin sponge, a proportion of M1 cells (CD86+) occupied over the majority of cells. However, in the case of kdES, M2 cell proportion (CD206+) was rather high and on the increase over time. Therefore, it could be confirmed that, when kdES is in vivo transplanted, the proportion of M2 cells is increased and may get involved in regeneration and wound healing.

### Example 12. Assessment of Tissue Restoration Pattern to Biodegradation Rate

In order to assess in vivo the importance of EDC/NHS used in the production of kdES, a group without EDC/NHS (kdECM hydrogel) and a group including EDC/NHS (kdES) were compared by the same method to confirm influence on tissue regeneration.

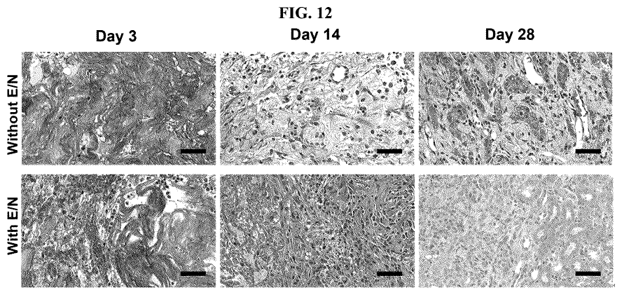

For this purpose, after transplantation of kdES or porous kdECM in the same animal model as Example 9, kdES degradation extent and healing conditions over time such as on day 3, day 14 and day 28 were investigated and shown in FIG. 12. In FIG. 12, the blue portion indicates a sponge transplanted site.

More particularly, the biodegradation rate was determined by comparing an area occupied by cells relative to the same area in a tissue photograph through ImageJ Software. A large area occupied by cells means that biodegradation proceeded much and a lot of tissues was regenerated.

As shown in FIG. 12, it could be understood that the blue area indicating the sponge transplanted site became quite thin on day 14, and therefore, degradation was proceeding to a certain extent. Further, in the case of kdES group on day 28, the degradation rate was delayed in a transplantation environment due to a bonding force between proteins improved through EDC/NHS, however, porous kdECM group showed remarkably rapid degradation of materials from 14 days and, when 28 days have passed, very irregular cell penetration was confirmed (scale bar: 200 um)

From the above results, although both of the groups are transplant materials using kidney-derived protein, it could be understood that the degradation rate in the body has significant influence upon a regeneration process.

The above description of the present invention is proposed for illustrative purpose, and it will be appreciated to those skilled in the art to which the present invention pertains that other specific variations or modifications are easily possible without altering the technical spirit or essential features of the present invention. Therefore, it should be understood that the examples described above are illustrative in all aspects and duly not limited thereto.

1. A method for production of a porous material for tissue regeneration and hemostasis, comprising the following steps:
(a) cutting a separated kidney tissue into sections and washing the same;
(b) treating the sections with a surfactant and agitating the same;
(c) removing DNA component residue within the kidney tissue;
(d) lyophilizing the kidney tissue free of the DNA component to thus obtain an extracellular matrix of decellularized kidney tissue;
(e) mixing the extracellular matrix of the decellularized kidney tissue with a cross-linking agent, followed by stirring and freezing the mixture; and
(f) washing the frozen mixture and lyophilizing the same to thus produce a porous material including the extracellular matrix of the decellularized kidney tissue.

2. The method according to claim 1, wherein the porous material is a sponge.

3. The method according to claim 1, wherein the step (b) is a step of treatment together with sodium chloride.

4. The method according to claim 1, wherein the cross-linking agent is one or more selected from the group consisting of 1-ethyl-3-(3-dimethylaminopropyl) carbodiimide hydrochloride (EDC) and N-hydroxysucinimide (NHS).

5. The method according to claim 1, wherein the freezing in the step (e) is conducted at −40 to −10° C. for 12 to 36 hours.

6. The method according to claim 4, wherein the cross-linking agent is used as a mixture of EDC solution: NHS solution=2 to 6:1 mole ratio.

7. The method according to claim 4, wherein the cross-linking agent is used as a mixture of EDC solution: NHS solution=1:0.5 to 2 mass ratio.

8. The method according to claim 1, wherein, in the step (e), a solution of the extracellular matrix of decellularized kidney tissue: the cross-linking agent solution=4 to 8:1 mass ratio are admixed, followed by agitating the same.

9. The method according to claim 1, wherein the porous material is used for reproduction and hemostasis of one or more tissues selected from the group consisting of kidney tissue, liver tissue, heart tissue, intestinal tissue, skin tissue, stomach tissue, pancreas tissue, brain tissue, thyroid tissue, lung tissue, and spleen tissue.

10. A porous material for tissue regeneration and hemostasis, comprising an extracellular matrix of decellularized kidney tissue prepared by the method according to claim 1, wherein the extracellular matrix of the decellularized kidney tissue has a structure consisting of a chemical cross-linking bond between proteins.

11. The porous material according to claim 10, wherein the porous material is a sponge.

12. The porous material according to claim 10, wherein the chemical cross-linking bond is formed by one or more cross-linking agents selected from the group consisting of 1-ethyl-3-(3-dimethylaminopropyl) carbodiimide hydrochloride (EDC) and N-hydroxysuccinimide (NHS).

13. The porous material according to claim 10, wherein the porous material has one or more among the following activities:
(i) reduction of a degradation rate;
(ii) hemostasis (bleeding stop) in damaged site of tissue;
(iii) tissue regeneration; and
(iv) increase in proportion of M2 macrophages.

14. The porous material according to claim 10, wherein the porous material is used for transplantation in vivo or as an external skin preparation.

15. The porous material according to claim 10, wherein the porous material is produced in one or more forms selected from the group consisting of gauze, sheet, patch and pad.

16. A method of treating a disease caused by tissue damage comprising:
administering a composition comprising the porous material for tissue regeneration and hemostasis prepared by the method according to claim 1 as an active ingredient to a subject in need thereof.

17. The method according to claim 16, wherein the disease is a disease caused by damage of any one or more tissues selected from the group consisting of kidney tissue, liver tissue, heart tissue, intestinal tissue, skin tissue, stomach tissue, pancreas tissue, brain tissuc. thyroid tissue. lung tissue, and spleen tissuc.

18. The method according to claim 16, wherein the composition is used for medical treatment of tissue damage sites; or for hemostasis and tissue regeneration in the case of tissue ablation or incision operation.
